(19) United States
(12) Patent Application Publication
(10) Pub. No.: US 2026/0294416 A1
(43) Pub. Date: Oct. 1, 2026

(54) LEFT ATRIAL APPENDAGE OCCLUSION DEVICE DELIVERY SYSTEMS

(71) Applicants: Medtronic, Inc., Minneapolis, MN (US); Covidien LP, Mansfield, MA (US); Medtronic CryoCath LP, Toronto (CA)

(72) Inventors: Junwei Li, Irvine, CA (US); Robert A. Pecor, Aliso Viejo, CA (US); Kevin R. Seifert, Forest Lake, MN (US); Thomas A. Anderson, New Hope, MN (US); Brian T. Howard, Minneapolis, MN (US); Bushan K. Purushothaman, Santa Rosa, CA (US); Nicolas Coulombe, Anjou (CA); Randal C. Schulhauser, Phoenix, AZ (US); Zhongping Yang, Woodbury, MN (US)

(21) Appl. No.: 19/479,255

(22) PCT Filed: Apr. 24, 2024
(86) PCT No.: PCT/US2024/026011

§ 371 Date: Oct. 27, 2025

Related U.S. Application Data
(60) Provisional application No. 63/499,056, filed on Apr. 28, 2023.

(51) Int. Cl. A61B 17/00

(52) U.S. Cl. CPC A61B 17/0057; A61B 17/00234; A61B 2017/00243; A61B 2017/00305; A61B 2017/00477; A61B 2017/0061; A61B 2017/00623; A61B 2017/00632; A61B 2017/00951

(57) ABSTRACT

In some examples, an example occlusion device delivery system according to aspects of the present disclosure may include an elongated shaft and a coupling mechanism for coupling an occlusion device. The elongated shaft may extend from a proximal shaft end to a distal shaft end. The coupling mechanism may be secured to the distal shaft end. The coupling mechanism may be configured to removably couple the elongated shaft to a coupling member of an occlusion device. The elongated shaft may be configured to deliver the occlusion device to an anatomical chamber.

This application claims the benefit of U.S. Provisional Application No. 63/499,056, filed 28 Apr. 2023, the entire contents of which is incorporated herein by reference.

### TECHNICAL FIELD

The present disclosure generally relates to left atrial appendage occlusion, and to systems for delivering devices for left atrial appendage occlusions.

### BACKGROUND

Atrial fibrillation may result in thrombus formation, which may cause embolic strokes. In nonvalvular atrial fibrillation (AF), thrombi typically occur in the left atrial appendage (LAA). The LAA is a pocket connected to the left atrium, which normally contracts with the left atrium.

The LAA may include features such as muscular ridges and folds formed on the interior surface of the LAA, which may promote blood pooling and stagnation within the LAA and, consequently, the formation of blood clots. Likewise, the relatively small size of the LAA may promote the formation of blood clots. Additionally, in AF the heart may beat rapidly and often with less force, which may not allow the LAA to adequately receive and expel fresh blood from within. If a blood clot escapes the LAA and enters the left atrium to be circulated throughout the body, there is a risk that the clot will cause an embolism and stroke. However, AF may cause the LAA to insufficiently contract, resulting in stagnant blood which may promote clot formation. Thrombi or clots released from the LAA may be associated with thromboembolic events.

LAA occlusion (LAAO) may be used to prevent thromboembolic events. For example, the LAA may be occluded to reduce or prevent formation of clots, or migration of clots from the LAA. Other procedures for preventing formation or migration of clots from the LAA include removal of the LAA, ligation or closure of the LAA, implanting a device within the LAA that prevents the flow of blood into and out of the LAA, and implanting a device within the LAA that functions as a filter to allow the flow of blood therethrough but prevent the movement of larger particles, such as clots, from exiting the LAA. Drug therapy may include administration of oral anticoagulants to reduce or prevent clot formation. Clinicians or patients may prefer an alternative or therapeutic treatment.

### SUMMARY

The present disclosure describes example systems for delivering occlusion devices for occluding an anatomical chamber, for example, to occlude a left atrial appendage of a patient.

An example occlusion device delivery system according to aspects of the present disclosure may include an elongated shaft and a coupling mechanism for coupling an occlusion device. The elongated shaft may extend from a proximal shaft end to a distal shaft end. The coupling mechanism may be secured to the distal shaft end. The coupling mechanism may be configured to removably couple the elongated shaft to a coupling member of an occlusion device. The elongated shaft may be configured to deliver the occlusion device to an anatomical chamber.

An example occlusion device delivery system according to aspects of the present disclosure may include an elongated shaft and a threaded bushing. The elongated shaft may extend from a proximal shaft end to a distal shaft end. The threaded bushing may be secured to the distal shaft end. The threaded bushing may be configured to removably couple the elongated shaft to a threaded coupling member of an occlusion device. The elongated shaft may be configured to deliver the occlusion device to an anatomical chamber.

An example method according to aspects of the present disclosure may include advancing an elongated shaft including a threaded bushing coupled to a threaded coupling member of an occlusion device through an anatomical structure of a patient to deliver the occlusion device within an anatomical chamber of the patient. The method may further include expanding the occlusion device to occlude at least a portion of the anatomical chamber. The method may further include uncoupling the elongated shaft from the occlusion device. The method may further include retracting the elongated shaft from the patient.

An example occlusion device delivery system according to aspects of the present disclosure may include an elongated shaft and a collet assembly. The elongated shaft may extend from a proximal shaft end to a distal shaft end. The collet assembly may be secured to the distal shaft end. The collet assembly may be configured to removably couple the elongated shaft to a protruding coupling member of an occlusion device. The elongated shaft may be configured to deliver the occlusion device to an anatomical chamber.

An example method according to aspects of the present disclosure may include advancing an elongated shaft including a collet assembly coupled to a protruding coupling member of an occlusion device through an anatomical structure of a patient to deliver the occlusion device within an anatomical chamber of the patient. The method may further include expanding the occlusion device to occlude at least a portion of the anatomical chamber. The method may further include uncoupling the elongated shaft from the occlusion device by releasing the protruding coupling member of the occlusion device from the collet assembly. The method may further include retracting the elongated shaft from the patient.

An example occlusion device delivery system according to aspects of the present disclosure includes an elongated shaft, an expandable tip, and a fill tube. The elongated shaft extending from a proximal shaft end to a distal shaft end. The expandable tip may be secured to the distal shaft end. The fill tube may be advanceable through the expandable tip. The expandable tip may be configured to be received within an interior of an attachment collar of an occlusion device. The fill tube may be configured to removably couple the elongated shaft to the occlusion device by causing the expandable tip to expand to engage with the interior of the attachment collar. The elongated shaft may be configured to deliver the occlusion device to an anatomical chamber.

An example method according to aspects of the present disclosure may include advancing an elongated shaft including an expandable tip coupled to an attachment collar of an occlusion device through an anatomical structure of a patient to deliver the occlusion device within an anatomical chamber of the patient. The method may further include expanding the occlusion device to occlude at least a portion of the anatomical chamber. The method may further include uncoupling the elongated shaft from the occlusion device by releasing the attachment collar of the occlusion device from the expandable tip. The method may further include retracting the elongated shaft from the patient.

An example occlusion device delivery system according to aspects of the present disclosure includes an elongated shaft, a clamp assembly, and a coupling sleeve. The elongated shaft may extend from a proximal shaft end to a distal shaft end. The clamp assembly may be secured to the distal shaft end and biased to radially expand in absence of an external constraint. The coupling sleeve may extend at least partly along the elongated shaft. The coupling sleeve may be movable relative to the clamp assembly. The coupling sleeve may be configured to removably couple the elongated shaft to the occlusion device by causing the clamp assembly to contract radially inward to clamp a portion of the occlusion device. The elongated shaft may be configured to deliver the occlusion device to an anatomical chamber.

An example method according to aspects of the present disclosure may include advancing an elongated shaft including a coupling sleeve covering a clamp assembly coupled to a portion of an occlusion device through an anatomical structure of a patient to deliver the occlusion device within an anatomical chamber of the patient. The method may further include expanding the occlusion device to occlude at least a portion of the anatomical chamber. The method may further include uncoupling the elongated shaft from the occlusion device by releasing the portion of the occlusion device from the clamp assembly. The method may further include retracting the elongated shaft from the patient.

The details of one or more examples are set forth in the accompanying drawings and the description below. Other features, objects, and advantages will be apparent from the description and drawings, and from the claims.

### BRIEF DESCRIPTION OF THE DRAWINGS

FIG. 1A is a partial side view of an example system including an elongated shaft and a threaded bushing for delivering an occlusion device.

FIG. 1B is a partial side view of the system of FIG. 1A with the occlusion device delivered in an anatomical chamber.

FIG. 2 is a partial view of a patient's heart showing a delivery site for the occlusion device of FIG. 1A.

FIG. 3 is a flowchart showing an example technique for occluding an anatomical chamber of a patient with the system of FIG. 1A.

FIG. 4A is a partial side view of an example system including an elongated shaft and a collet assembly for delivering an occlusion device.

FIG. 4B is a partial side view of the system of FIG. 4A with the occlusion device secured to the collet assembly.

FIG. 5 is a flowchart showing an example technique for occluding an anatomical chamber of a patient with the system of FIG. 4A.

FIG. 6A is a partial cross-sectional view of an example system including an elongated shaft and an expandable tip for delivering an occlusion device.

FIG. 6B is a partial cross-sectional view of the system of FIG. 6A with the occlusion device secured to the expandable tip.

FIG. 7 is a flowchart showing an example technique for occluding an anatomical chamber of a patient with the system of FIG. 6A.

FIG. 8A is a partial cross-sectional view of an example system including an elongated shaft and a clamp assembly for delivering an occlusion device.

FIG. 8B is a partial cross-sectional view of the system of FIG. 8A with the occlusion device released from the clamp assembly.

FIG. 9 is a flowchart showing an example technique for occluding an anatomical chamber of a patient with the system of FIG. 8A.

### DETAILED DESCRIPTION

In general, aspects of the present disclosure relate to left atrial appendage occlusion, and to systems for delivering devices for left atrial appendage occlusions.

Atrial fibrillation may result in thrombus formation, which may cause embolic strokes. In nonvalvular atrial fibrillation (AF), thrombi typically occur in the left atrial appendage (LAA). The LAA is a pocket connected to the left atrium, which normally contracts with the left atrium.

The LAA may include features such as muscular ridges and folds formed on the interior surface of the LAA, which may promote blood pooling and stagnation within the LAA and, consequently, the formation of blood clots. Likewise, the relatively small size of the LAA may promote the formation of blood clots. Additionally, in AF the heart may beat rapidly and often with less force, which may not allow the LAA to adequately receive and expel fresh blood from within. If a blood clot escapes the LAA and enters the left atrium to be circulated throughout the body, there is a risk that the clot will cause an embolism and stroke. However, AF may cause the LAA to insufficiently contract, resulting in stagnant blood which may promote clot formation. Thrombi or clots released from the LAA may be associated with thromboembolic events.

LAA occlusion (LAAO) may be used to prevent thromboembolic events. For example, the LAA may be occluded to reduce or prevent formation of clots, or migration of clots from the LAA. Other procedures for preventing formation or migration of clots from the LAA include removal of the LAA, ligation or closure of the LAA, implanting a device within the LAA that prevents the flow of blood into and out of the LAA, and implanting a device within the LAA that functions as a filter to allow the flow of blood therethrough but prevent the movement of larger particles, such as clots, from exiting the LAA. Drug therapy may include administration of oral anticoagulants to reduce or prevent clot formation. Clinicians or patients may prefer an alternative or therapeutic treatment.

Systems and methods according to the present disclosure may be used to deliver an occlusion device to an anatomical chamber. The anatomical chamber may be the LAA. For example, systems and methods according to the present disclosure may allow delivering an expandable occlusion device to the anatomical chamber, and to deliver biogel to cause the occlusion device to expand.

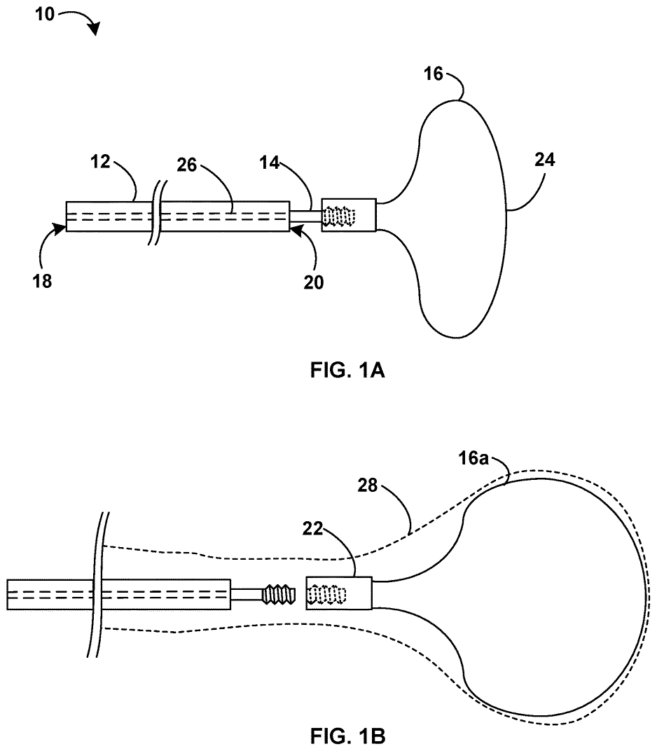

FIG. 1A is a partial side view of an example system 10 including an elongated shaft 12 and a threaded bushing 14 for delivering an occlusion device 16. Elongated shaft 12 may extend from a proximal shaft end 18 to a distal shaft end 20. Elongated shaft 12 may include at least one polymer. For example, the polymer may include any suitable medical, clinical, or biocompatible polymer. Elongated shaft 12 may be flexible or maneuverable, for example, through an anatomical structure of a patient. Elongated shaft 12 may be sufficiently stiff to transmit force, pressure, or torque to distal shaft end 20, for example, to occlusion device 16 secured to distal shaft end 20.

Occlusion device 16 may be removably securable to elongated shaft 12 by threaded bushing 14. Threaded bushing 14 may be secured to distal shaft end 20. Threaded bushing 14 may be configured to removably couple elongated shaft 12 to a threaded coupling member 22 of occlusion device 16. For example, threaded coupling member 22 may be screwed onto or within threaded bushing 14 by relative rotation between occlusion device 16 and elongated shaft 12. Threaded bushing 14 may include at least one of a metal, an alloy, or a polymer. Threaded bushing 14 may be relatively rigid, and sufficiently stiff to accommodate relative rotational engagement of threaded bushing 14 and threaded coupling member 22. Threaded coupling member 22 may include one or materials similar to those described with reference to threaded bushing 14.

Threaded bushing 14 may be secured to distal shaft end 20 by at least one of an adhesive, a weld, or an overmold. Threaded coupling member 22 may be secured to occlusion device 16 by at least one of an adhesive, a weld, or an overmold.

In some examples, threaded bushing 14 is configured to be threaded within an interior of threaded coupling member 22 of occlusion device 16. In other examples, threaded bushing 14 is configured to be threaded about an exterior of threaded coupling member 22 of occlusion device 16. For example, threaded bushing 14 may be wider than threaded coupling member 22, threaded bushing 14 may define threads within an interior of threaded bushing 14, and threaded coupling member may define threads about an exterior surface of threaded coupling member 22.

Occlusion device 16 may include an expanding member 24 configured to expand in response to receiving a volume of biogel. Expanding member 24 may include a device frame. The device frame may include at least one of a film layer, a mesh layer, a woven layer, or a non-woven layer.

Elongated shaft 12 may define a shaft lumen 26 extending from the proximal shaft end to the distal shaft end. The shaft lumen may be configured to deliver biogel to occlusion device 16 to cause expansion of occlusion device 16.

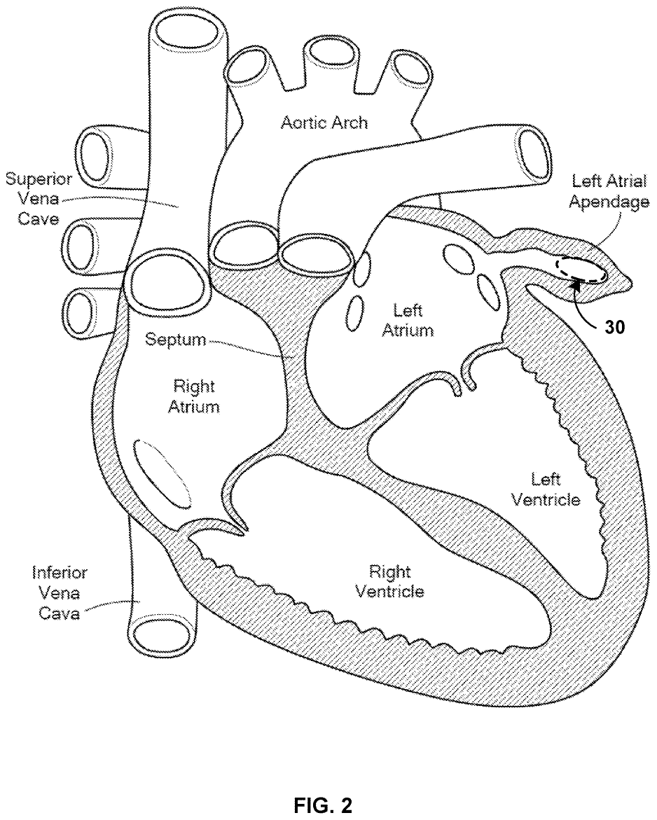

FIG. 1B is a partial side view of system 10 of FIG. 1A with occlusion device 16 delivered in an anatomical chamber 28 and in an expanded configuration 16a. Anatomical chamber 28 may be a left atrial appendage, as described with reference to FIG. 2.

Elongated shaft 12 may be configured to deliver occlusion device 16 to anatomical chamber 28, and may be further configured to control expansion of occlusion device 16 by delivering biogel. In other examples, an element, catheter, or cannula other than elongated shaft 12 may be used to deliver biogel, and elongated shaft 12 may be used to deliver occlusion device 16. In some examples, the other element, catheter, or cannula may be deliverable through shaft lumen 26 of elongated shaft.

As occlusion device 16 expands within anatomical chamber 28, blood may be displaced from anatomical chamber 28. In some examples, elongated shaft 12 may facilitate removal of the displaced blood. For example, shaft lumen 26 may be a first shaft lumen, and elongated shaft 12 may define a second shaft lumen (not shown) extending from proximal shaft end 18 to distal shaft end 20. The second shaft lumen may be configured to retrieve blood from anatomical chamber 28 in response to the expansion of occlusion device 16 in the anatomical chamber. The second shaft lumen may be similar to shaft lumen 26.

FIG. 2 is a partial view of a patient's heart showing a delivery site 30 for occlusion device 16 of FIG. 1A. A clinician may navigate or maneuver occlusion device 16 secured to elongated shaft 12 of system 10 to delivery site 30, and decouple occlusion device 16 at delivery site 30. For example, the clinician may rotate elongated shaft 12 relative to occlusion device 16 to cause threaded bushing 14 to unscrew from threaded coupling member 22. In some examples, the clinician may rotate elongated shaft 12 relative to occlusion device 16 after expansion of occlusion device 16 as expanded occlusion device 16 may frictionally engage the delivery site in the patient to allow relative rotation of shaft 12 and occlusion device 16.

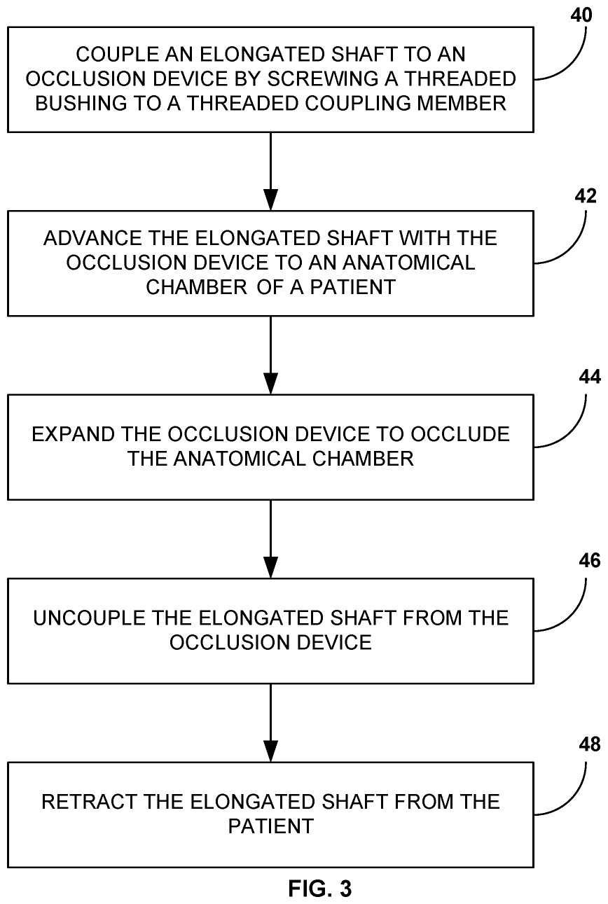

FIG. 3 is a flowchart showing an example technique for occluding an anatomical chamber of a patient with the system of FIG. 1A. The example technique of FIG. 3 is described with reference to system 10, however, the example technique may be performed with any suitable system.

The technique may include coupling elongated shaft 12 to occlusion device 16 by screwing threaded bushing 14 secured to elongated shaft 12 to threaded coupling member 22 of occlusion device 16 (40). Coupling the elongated shaft 12 to occlusion device 16 (40) may include screwing threaded bushing 16 within an interior of threaded coupling member 22 of occlusion device 16. In other examples, coupling elongated shaft 12 to occlusion device 16 may include screwing threaded bushing 16 about an exterior of threaded coupling member 22. In some examples, a manufacturer of occlusion device 16 may provide the occlusion device coupled to elongated shaft, so that an implanter need not do so prior to implantation.

The technique may further include advancing elongated shaft 12 through an anatomical structure of a patient to deliver occlusion device 16 within anatomical chamber 28 of the patient (42). Anatomical chamber 28 may be a left atrial appendage.

The technique illustrated by FIG. 3 may further include expanding occlusion device 16 to occlude at least a portion of anatomical chamber 28 (44). Expanding occlusion device (44) may include delivering a biogel within an interior of occlusion device 16 to cause occlusion device 16 to expand. Delivering the biogel may include transporting the biogel from a biogel source through shaft lumen 26 of elongated shaft 12 to the interior of occlusion device 16.

In some examples, the technique includes delivering the biogel after expanding occlusion device 16 (44). For example, occlusion device 16 may be mechanically expanded, for example, with an actuating mechanism, followed by delivering of biogel within expanded occlusion device 16.

The technique may further include withdrawing blood from the anatomical chamber 28. For example, during or after the expanding (44), blood may be displaced from anatomical chamber 28, and such blood may be withdrawn, e.g., by active or passive flow through elongated shaft 12.

The technique may further include uncoupling elongated shaft 12 from occlusion device 16 (46). The technique may further include retracting elongated shaft 12 from the patient (48), for example, leaving occlusion device 16 within anatomical chamber 28.

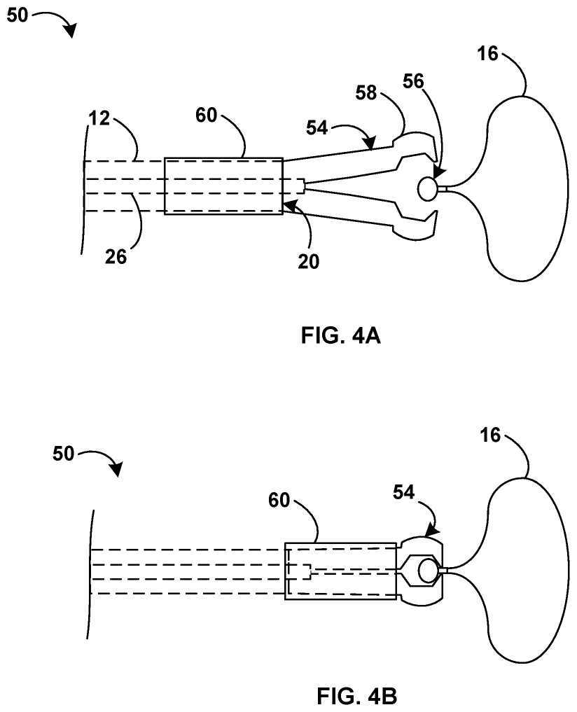

FIG. 4A is a partial side view of an example system 50 including elongated shaft 12 and a collet assembly 54 for delivering occlusion device 16.

Collet assembly 54 may be secured to distal shaft end 20. Collet assembly 54 may include at least one of a metal, an alloy, or a polymer. Collet assembly 54 may include a plurality of collet members 58 biased to expand radially in the absence of an external constraint and contract radially in the presence of the external constraint. Collet assembly 54 may be secured to distal shaft end by 20 at least one of an adhesive, a weld, or an overmold.

Collet assembly 54 may be configured to removably couple elongated shaft 12 to a protruding coupling member 56 of occlusion device 16. Protruding coupling member 56 may include a ball, a bead, or some other structure receivable or holdable in collet assembly 54. Coupling member 56 may include a necked portion, e.g., including a smaller diameter or other dimension transverse to a longitudinal axis of occlusion device at a first portion more distal than a second portion.

System 50 may further include a coupling sleeve 60 extending at least partly along elongated shaft 12. Coupling sleeve 54 includes at least one of a polymer, a metal, or an alloy. Coupling sleeve 60 may be coextensive with elongated shaft 12, or may extend partially along elongated shaft 12. In some examples, an actuating cable may be coupled to coupling sleeve 60 to facilitate translational movement of coupling sleep relative to elongated shaft 12. Thus, coupling sleeve 60 may be movable relative to the collet assembly. Coupling sleeve 60 is configured to contract the expandable collet assembly radially inward in response to advancing the coupling sleeve over a portion of the collet assembly.

FIG. 4B is a partial side view of system 50 of FIG. 4A with occlusion device 16 secured to collet assembly 54. For example, an inner surface of coupling sleeve 54 may contact an outer surface of collet assembly 54 and apply a constraint to collet assembly 54, when coupling sleeve 54 is moved toward and over collet assembly 54. Retracting coupling sleeve 54 away from collet assembly 54 may remove the constraint and allow collet assembly 54 to expand in response to an internal bias of collet assembly 54.

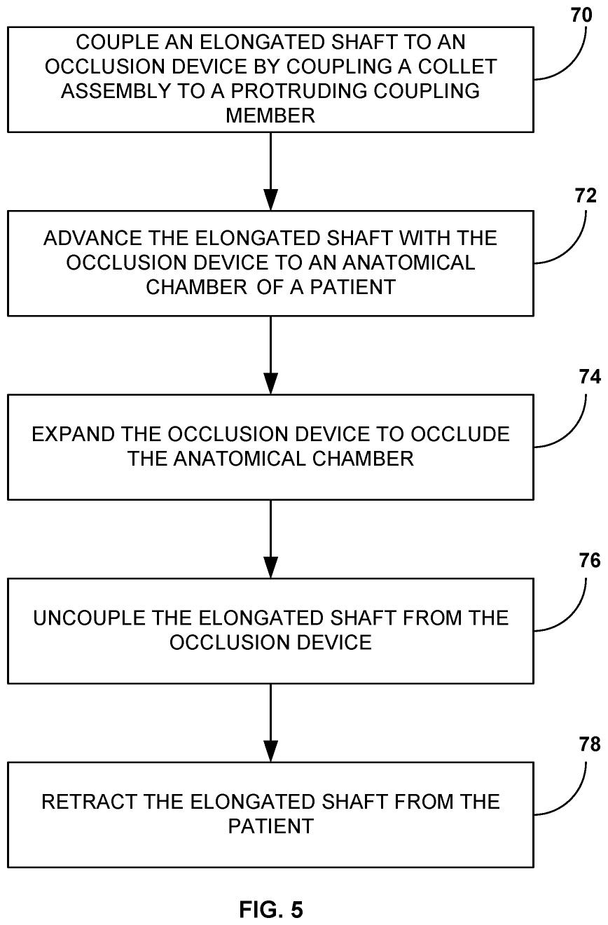

FIG. 5 is a flowchart showing an example technique for occluding an anatomical chamber of a patient with the system of FIG. 4A. The example technique of FIG. 5 is described with reference to system 50, however, the example technique may be performed with any suitable system.

The technique may include coupling elongated shaft 12 to occlusion device 16 by coupling collet assembly 54 secured to elongated shaft 12 to a protruding coupling member 56 of occlusion device 16 (70). Coupling elongated shaft 12 to occlusion device 16 (70) may include radially contracting collet assembly 54 about protruding coupling member 56 of occlusion device 16. In some examples, a manufacturer of occlusion device 16 may provide the occlusion device coupled to elongated shaft, so that an implanter need not do so prior to implantation.

The technique may further include advancing elongated shaft 12 through an anatomical structure of a patient to deliver occlusion device 16 within anatomical chamber 28 of the patient (72). Anatomical chamber 28 may be a left atrial appendage.

The technique may further include expanding occlusion device 16 to occlude at least a portion of anatomical chamber 28 (74). Expanding occlusion device (74) may include delivering a biogel within an interior of occlusion device 16 to cause occlusion device 16 to expand. Delivering the biogel may include transporting the biogel from a biogel source through shaft lumen 26 of elongated shaft 12 to the interior of occlusion device 16.

In some examples, the technique includes delivering the biogel after expanding occlusion device 16 (74). For example, occlusion device 16 may be mechanically expanded, for example, with an actuating mechanism, followed by delivering of biogel within expanded occlusion device 16.

The technique may further include withdrawing blood from the anatomical chamber 28. For example, during or after the expanding (74), blood may be displaced from anatomical chamber 28, and such blood may be withdrawn.

The technique may further include uncoupling elongated shaft 12 from occlusion device 16 (74). The uncoupling (74) may include releasing protruding coupling member 56 of occlusion device 16 from collet assembly 54. The uncoupling elongated shaft 12 from occlusion device 16 may include radially expanding collet assembly 54 to release protruding coupling member 56 of occlusion device 16. For example, coupling sleeve 60 may be retracted away from collet assembly 54, allowing collet assembly 54 to expand radially in response to an internal bias. In turn, collet members 58 may move away from each other to release protruding coupling member 56.

The technique may further include retracting elongated shaft 12 from the patient (78), for example, leaving occlusion device 16 within anatomical chamber 28.

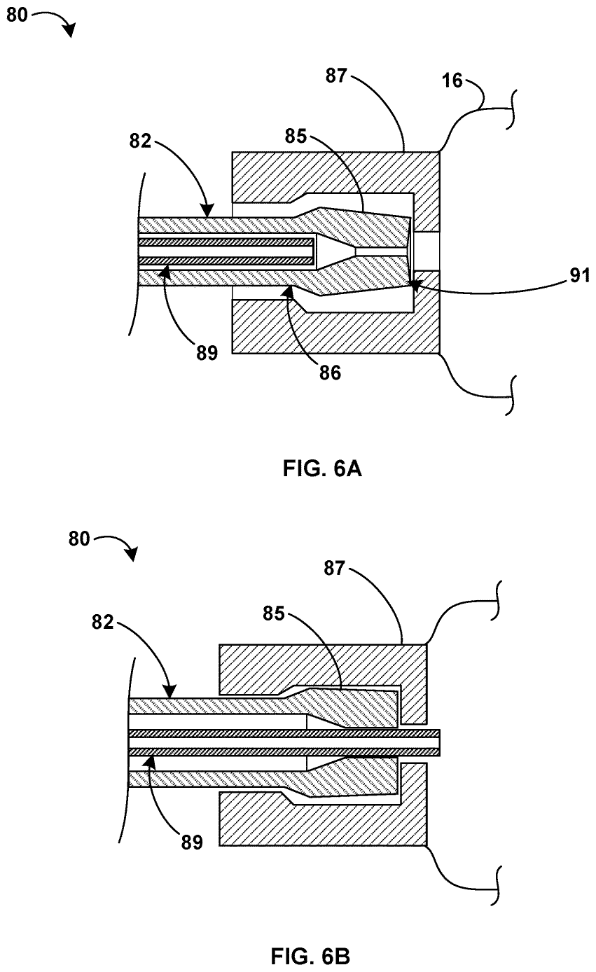

FIG. 6A is a partial cross-sectional view of an example system 80 including an elongated shaft 82 and an expandable tip 85 for delivering occlusion device 16. Elongated shaft 82 may be similar in shape and construction to elongated shaft 12 described with reference to FIGS. 1A and 1B, but differ in the presence of expandable tip 85. Elongated shaft 82 may extend from a proximal shaft end (not shown) to a distal shaft end 86. Elongated shaft 82 may be configured to deliver occlusion device 16 to anatomical chamber 28.

Expandable tip 85 may integrally extend from distal shaft end 86, or may be a distinct element and secured to distal shaft end 86. Expandable tip 85 may be secured to distal shaft end 82 by at least one of an adhesive, a weld, or an overmold. Expandable tip 85 may be configured to be received within an interior of an attachment collar 87 of occlusion device 16. Expandable tip 85 may include at least one of a metal, an alloy, or a polymer, and include a single expandable part, or a plurality of expandable parts that separate from each other. Expandable tip 85 may include one or more of a ring, a cylinder, a claw, a collet, a prong, or another suitable expanding element.

Attachment collar 87 may include at least one of a metal, an alloy, or a polymer. Attachment collar 87 may be relatively stiffer or harder than expandable tip 85, such that attachment collar 87 substantially retains its shape in response to expansion of expandable tip 85. In other examples, attachment collar 87 may be at least partially expandable, and expand in response to expansion of expandable tip 85, while remaining in engagement with expandable tip 85.

System 80 further includes a fill tube 89. Fill tube 89 may be advanceable through expandable tip 85, for example, through a distal opening of expandable tip 85. Fill tube 89 may be configured to removably couple elongated shaft 82 to occlusion device 16 by causing expandable tip 85 to expand to engage with the interior of attachment collar 87. Fill tube 89 may include at least one of a metal, an alloy, or a polymer. While advancing fill tube 89 toward or through expandable tip 85 may cause expandable tip 85 to expand, retracting fill tube 89 away from expandable tip 85 may cause expandable tip 85 to contract, for example, in response to an internal bias of expandable tip 85, or in response to removal of an expanding stress exerted by fill tube 89 on expandable tip 85. Expandable tip 85 may include a tip end portion 91 configured to expand radially in response to an introduction of fill tube 89 within an inter-tip spacing of tip end portion 91 and biased to contract radially in the absence of fill tube 89. Tip end portion 91 may include a plurality of tip members.

FIG. 6B is a partial cross-sectional view of system 80 of FIG. 6A with occlusion device 16 secured to expandable tip 85. In the configuration of FIG. 6B, fill tube 89 is advanced distally through expandable tip 85, causing expandable tip 85 to expand against the interior of attachment collar 87.

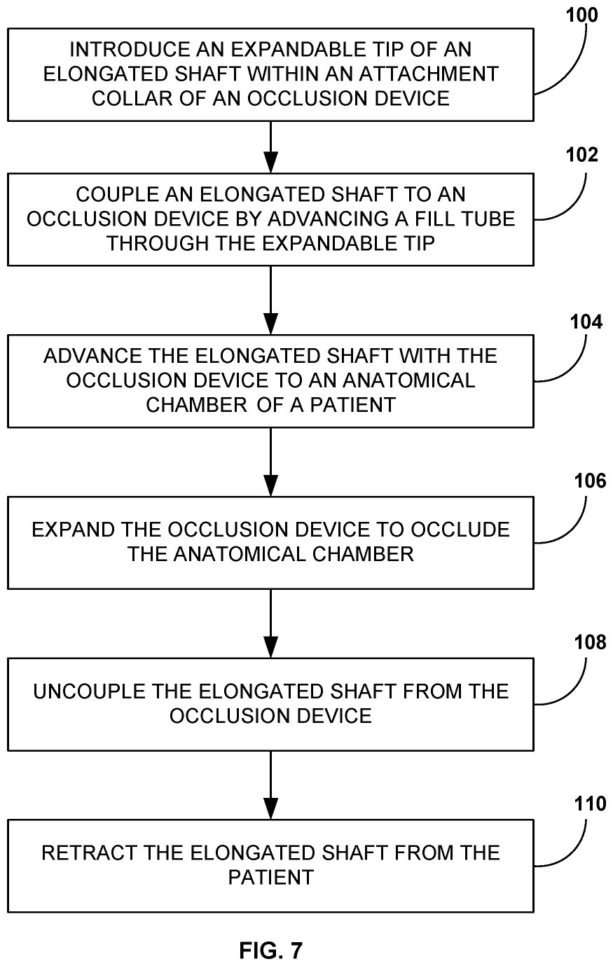

FIG. 7 is a flowchart showing an example technique for occluding anatomical chamber 28 of a patient with system 80 of FIG. 6A. The example technique of FIG. 7 is described with reference to system 80, however, the example technique may be performed with any suitable system.

The technique may include introducing expandable tip 85 secured to elongated shaft 82 within an interior of attachment collar 87 of occlusion device 16 (100). The technique may further include coupling elongated shaft 82 to occlusion device 16 by advancing fill tube 89 through expandable tip 85 to cause expandable tip 85 to contact the interior of attachment collar 87 of occlusion device 16 (102). Coupling elongated shaft 82 to occlusion device 16 (102) may include radially expanding expandable tip 85 within the interior of attachment collar 87. In some examples, a manufacturer of occlusion device 16 may provide the occlusion device coupled to elongated shaft 82, so that an implanter need not do so prior to implantation.

The technique may further include advancing elongated shaft 82 through an anatomical structure of a patient to deliver occlusion device 16 within anatomical chamber 28 of the patient (104). The anatomical chamber may be a left atrial appendage.

The technique may further include expanding occlusion device 16 to occlude at least a portion of anatomical chamber 28 (106). Expanding occlusion device 16 (106) may include delivering a biogel within an interior of occlusion device 16 to cause occlusion device 16 to expand. The biogel may be delivered through a lumen of elongated shaft 82, or through a lumen of fill tube 89. The technique may include delivering a biogel within an interior of occlusion device 16 after the expanding (106). Delivering the biogel may include transporting the biogel from a biogel source through a shaft lumen of elongated shaft 82 or a lumen of fill tube 89 to the interior of the occlusion device.

The method may further include withdrawing blood from anatomical chamber 28. For example, during or after the expanding (106), blood may be displaced from anatomical chamber 28, and such blood may be withdrawn.

The technique may further include uncoupling elongated shaft 82 from occlusion device 16 (108). The uncoupling (108) may include releasing attachment collar 87 of occlusion device 16 from expandable tip 85. For example, uncoupling elongated shaft 82 from occlusion device 16 (108) may include radially contracting expandable tip 85 to release attachment collar 87 of the occlusion device 16 from expandable tip 85.

The technique may further include retracting elongated shaft 82 from the patient (110), for example, leaving occlusion device 16 within anatomical chamber 28.

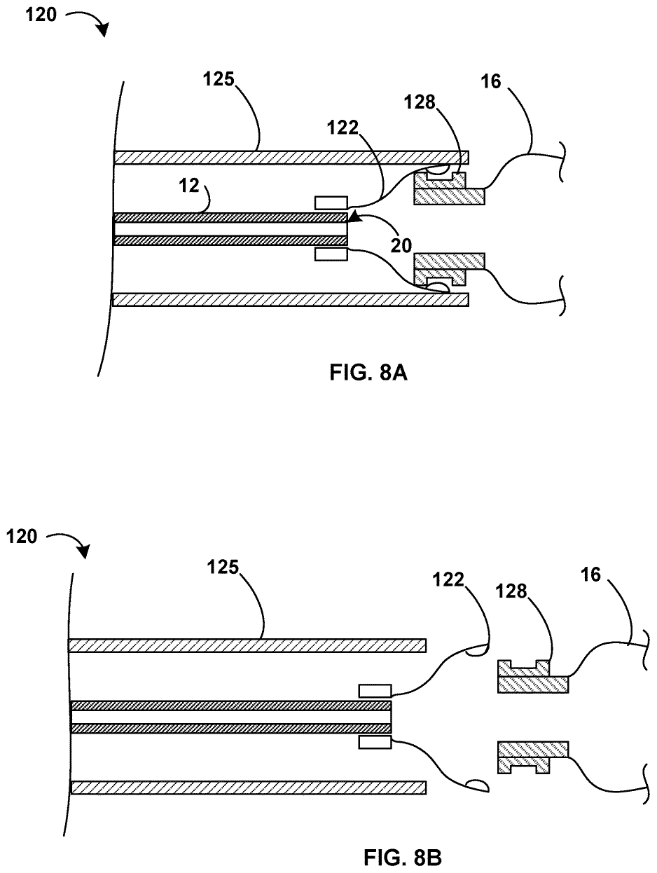

FIG. 8A is a partial cross-sectional view of an example system 120 including elongated shaft 12 and a clamp assembly 122 for delivering occlusion device 16. System 120 further includes a coupling sleeve 125. Coupling sleeve 125 may be similar to coupling sleeve 85.

Clamp assembly 122 may be secured to or adjacent distal shaft end 12 and biased to radially expand in absence of an external constraint. Clamp assembly may include a plurality of prongs biased to expand radially outward in the absence of the external constraint. Each prong of the plurality of prongs include a beaded tip. Clamp assembly 122 may include at least one of a metal, an alloy, or a polymer. Clamp assembly 122 may be secured to distal shaft end 20 by at least one of an adhesive, a weld, or an overmold.

Coupling sleeve 125 may extend at least partly along elongated shaft 12. Coupling sleeve 125 may include at least one of a metal, an alloy, or a polymer. Coupling sleeve 125 may be movable relative to clamp assembly 122. Coupling sleeve 125 may be configured to removably couple elongated shaft 12 to occlusion device 16 by causing clamp assembly 122 to contract radially inward to clamp a portion 128 of occlusion device 16. Portion 128 of the occlusion device may include an attachment collar, for example, an attachment collar defining a slot or a recess. The slot or recess may circumferentially extend about the attachment collar. The attachment collar may be similar to attachment collar 87.

FIG. 8B is a partial cross-sectional view of system 120 of FIG. 8A with occlusion device 16 released from clamp assembly 122. In the configuration of FIG. 8B, coupling sleeve 125 is retracted away from protrusion 128, and from clamp assembly 122, allowing clamp assembly 122 to expand radially outward, for example, in response to an internal bias. The radial outward expansion of clamp assembly 122 releases portion 128 from clamp assembly 122.

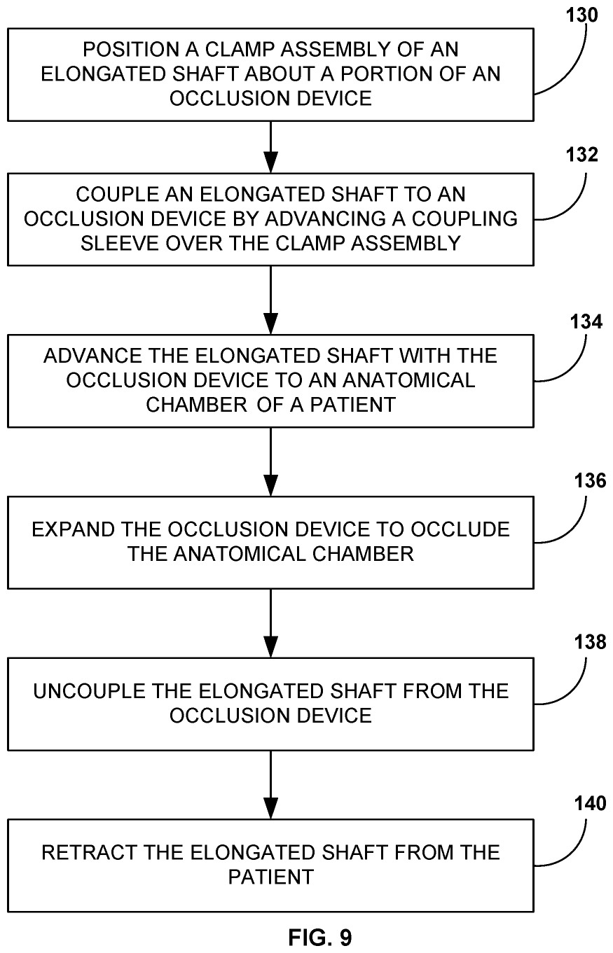

FIG. 9 is a flowchart showing an example technique for occluding an anatomical chamber of a patient with system 120 of FIG. 8A. The example technique of FIG. 9 is described with reference to system 120, however, the example technique may be performed with any suitable system.

The technique may include positioning clamp assembly 122 secured to elongated shaft 12 about portion 128 of occlusion device 16 (130). The technique may further include coupling elongated shaft 12 to occlusion device 16 by advancing coupling sleeve 125 over clamp assembly 122 to cause clamp assembly 122 to clamp portion 128 of occlusion device 16 (132). In some examples, a manufacturer of occlusion device 16 may provide the occlusion device coupled to elongated shaft 12, so that an implanter need not do so prior to implantation.

The technique may further include advancing elongated shaft 12 through an anatomical structure of a patient to deliver the occlusion device within anatomical chamber 28 of the patient (134). Anatomical chamber 28 may be a left atrial appendage.

The technique may further include expanding occlusion device 16 to occlude at least a portion of anatomical chamber 28 (136). Expanding occlusion device 16 (136) may include delivering a biogel within an interior of occlusion device 16 to cause occlusion device 16 to expand. Biogel may be delivered within an interior of occlusion device 16 after expanding (136). Delivering the biogel may include transporting the biogel from a biogel source through a shaft lumen of the elongated shaft 12 to the interior of occlusion device 16.

The method may further include withdrawing blood from anatomical chamber 28. For example, during or after the expanding (106), blood may be displaced from anatomical chamber 28, and such blood may be withdrawn.

The technique may further include uncoupling elongated shaft 12 from occlusion device 16 by releasing portion 128 of occlusion device 16 from clamp assembly 122 (138). The uncoupling (108) may include retracting coupling sleeve 125 from clamp assembly 12 to allow clamp assembly 122 to expand and release portion 128 of occlusion device 16.

The technique may further include retracting elongated shaft 12 from the patient (140), for example, leaving occlusion device 16 within anatomical chamber 28.

The following list of enumerated clauses describes examples according to the present disclosure.
Clause 1: An occlusion device delivery system including: an elongated shaft extending from a proximal shaft end to a distal shaft end; and a threaded bushing secured to the distal shaft end, where the threaded bushing is configured to removably couple the elongated shaft to a threaded coupling member of an occlusion device, and where the elongated shaft is configured to deliver the occlusion device to an anatomical chamber.
Clause 2: The system of clause 1, where the elongated shaft includes at least one polymer, and where the threaded bushing includes at least one of a metal, an alloy, or a polymer.
Clause 3: The system of clauses 1 or 2, where the anatomical chamber is a left atrial appendage.
Clause 4: The system of any of clauses 1 to 3, where the threaded bushing is configured to be threaded within an interior of the threaded coupling member of the occlusion device.
Clause 5: The system of any of clauses 1 to 3, where the threaded bushing is configured to be threaded about an exterior of the threaded coupling member of the occlusion device.
Clause 6: The system of any of clauses 1 to 5, where the threaded bushing is secured to the distal shaft end by at least one of an adhesive, a weld, or an overmold.
Clause 7: The system of any one of clauses 1 to 6, further including the occlusion device, where the occlusion device includes an expanding member configured to expand in response to receiving a volume of biogel.
Clause 8: The system of clause 7, where the expanding member includes a device frame, and where the device frame includes at least one of a film layer, a mesh layer, a woven layer, or a non-woven layer.
Clause 9: The system of any one of clauses 1 to 8, where the elongated shaft defines a shaft lumen extending from the proximal shaft end to the distal shaft end, and where the shaft lumen is configured to deliver biogel to the occlusion device to cause expansion of the occlusion device.
Clause 10: The system of clause 9, where the shaft lumen is a first shaft lumen, where the elongated shaft defines a second shaft lumen extending from the proximal shaft end to the distal shaft end, and where the second shaft lumen is configured to retrieve blood from an anatomical chamber in response to the expansion of the occlusion device in the anatomical chamber.
Clause 11: A method including: advancing an elongated shaft including a threaded bushing coupled to a threaded coupling member of an occlusion device through an anatomical structure of a patient to deliver the occlusion device within an anatomical chamber of the patient; expanding the occlusion device to occlude at least a portion of the anatomical chamber; uncoupling the elongated shaft from the occlusion device by unscrewing the threaded bushing from a threaded coupling member of the occlusion device; and retracting the elongated shaft from the patient.
Clause 12: The method of clause 11, where expanding the occlusion device includes delivering a biogel within an interior of the occlusion device to cause the occlusion device to expand.
Clause 13: The method of clause 11, further including delivering a biogel within an interior of the occlusion device after expanding the occlusion device.
Clause 14: The method of clause 12 or 13, where delivering the biogel includes transporting the biogel from a biogel source through a shaft lumen of the elongated shaft to the interior of the occlusion device.
Clause 15: The method of any of clauses 11 to 14, further including withdrawing blood from the anatomical chamber.
Clause 16: The method of clause 15, where the shaft lumen of the elongated shaft is a first shaft lumen, and where withdrawing the blood includes transporting the blood from the anatomical chamber through a second shaft lumen of the elongated shaft.
Clause 17: The method of any of clauses 11 to 16, where the anatomical chamber is a left atrial appendage.
Clause 18: The method of any of clauses 11 to 17, further including, before the advancing, coupling the occlusion device to the elongated shaft by screwing the threaded bushing to the threaded coupling member of the occlusion device.
Clause 19: The method of clause 18, where coupling the elongated shaft to the occlusion device includes screwing the threaded bushing within an interior of the threaded coupling member of the occlusion device.
Clause 20: The method of clause 18, where coupling the elongated shaft to the occlusion device includes screwing the threaded bushing about an exterior of the threaded coupling member of the occlusion device.
Clause 21: An occlusion device delivery system including: an elongated shaft extending from a proximal shaft end to a distal shaft end; and a collet assembly secured to the distal shaft end, where the collet assembly is configured to removably couple the elongated shaft to a protruding coupling member of an occlusion device, and where the elongated shaft is configured to deliver the occlusion device to an anatomical chamber.
Clause 22: The system of clause 21, where the elongated shaft includes at least one polymer, and where the collet assembly includes at least one of a metal, an alloy, or a polymer.
Clause 23: The system of clauses 21 or 22, where the anatomical chamber is a left atrial appendage.
Clause 24: The system of any of clauses 21 to 23, where the collet assembly includes a plurality of collet members biased to expand radially in the absence of an external constraint and contract radially in the present of the external constraint.
Clause 25: The system of any of clauses 21 to 24, where the collet assembly is secured to the distal shaft end by at least one of an adhesive, a weld, or an overmold.
Clause 26: The system of any of clauses 21 to 25, further including a coupling sleeve extending at least partly along the elongated shaft, where the coupling sleeve is movable relative to the collet assembly, and where the coupling sleeve is configured to contract the expandable collet assembly radially inward in response to advancing the coupling sleeve over a portion of the collet assembly.
Clause 27: The system of clause 26, where the coupling sleeve includes at least one of a polymer, a metal, or an alloy.
Clause 28: The system of any one of clauses 21 to 27, further including the occlusion device, where the occlusion device includes an expanding member configured to expand in response to receiving a volume of biogel.
Clause 29: The system of clause 28, where the expanding member includes a device frame, and where the device frame includes at least one of a film layer, a mesh layer, a woven layer, or a non-woven layer.
Clause 30: The system of any one of clauses 21 to 29, where the elongated shaft defines a shaft lumen extending from the proximal shaft end to the distal shaft end, and where the shaft lumen is configured to deliver biogel to the occlusion device to cause expansion of the occlusion device.
Clause 31: The system of clause 30, where the shaft lumen is a first shaft lumen, where the elongated shaft defines a second shaft lumen extending from the proximal shaft end to the distal shaft end, and where the second shaft lumen is configured to retrieve blood from an anatomical chamber in response to the expansion of the occlusion device in the anatomical chamber.
Clause 32: A method including: advancing an elongated shaft including a collet assembly coupled to a protruding coupling member of an occlusion device through an anatomical structure of a patient to deliver the occlusion device within an anatomical chamber of the patient; expanding the occlusion device to occlude at least a portion of the anatomical chamber; uncoupling the elongated shaft from the occlusion device by releasing the occlusion device from the collet assembly; and retracting the elongated shaft from the patient.
Clause 33: The method of clause 32, where expanding the occlusion device includes delivering a biogel within an interior of the occlusion device to cause the occlusion device to expand.
Clause 34: The method of clause 32, further including delivering a biogel within an interior of the occlusion device after expanding the occlusion device.
Clause 35: The method of clauses 33 or 34, where delivering the biogel includes transporting the biogel from a biogel source through a shaft lumen of the elongated shaft to the interior of the occlusion device.
Clause 36: The method of any of clauses 32 to 35, further including withdrawing blood from the anatomical chamber.
Clause 37: The method of clause 36, where the shaft lumen of the elongated shaft is a first shaft lumen, and where withdrawing the blood includes transporting the blood from the anatomical chamber through a second shaft lumen of the elongated shaft.
Clause 38: The method of any of clauses 32 to 37, where the anatomical chamber is a left atrial appendage.
Clause 39: The method of any of clauses 32 to 38, further including, before the advancing, coupling the elongated shaft to the occlusion device by coupling the collet assembly secured to the elongated shaft to the protruding coupling member of the occlusion device.
Clause 40: The method of clause 39, where coupling the elongated shaft to the occlusion device includes radially contracting the collet assembly about the protruding coupling member of the occlusion device, and where uncoupling the elongated shaft from the occlusion device includes radially expanding the collet assembly to release the protruding member of the occlusion device.
Clause 41: An occlusion device delivery system including: an elongated shaft extending from a proximal shaft end to a distal shaft end; an expandable tip secured to the distal shaft end; and a fill tube advanceable through the expandable tip, where the expandable tip is configured to be received within an interior of an attachment collar of an occlusion device, where the fill tube is configured to removably couple the elongated shaft to the occlusion device by causing the expandable tip to expand to engage with the interior of the attachment collar, and where the elongated shaft is configured to deliver the occlusion device to an anatomical chamber.
Clause 42: The system of clause 41, where the elongated shaft includes at least one polymer, where the expandable tip includes at least one of a metal, an alloy, or a polymer, where the attachment collar includes at least one of a metal, an alloy, or a polymer, and where the fill tube includes at least one of a metal, an alloy, or a polymer.
Clause 43: The system of clauses 41 or 42, where the anatomical chamber is a left atrial appendage.
Clause 44: The system of any of clauses 41 to 43, where the expandable tip includes a tip end portion configured to expand radially in response to an introduction of the fill tube within an inter-tip spacing and biased to contract radially in the absence of the fill tube.
Clause 45: The system of clause 44, where the tip end portion includes a plurality of tip members.
Clause 46: The system of any of clauses 41 to 45, where the expandable tip is secured to the distal shaft end by at least one of an adhesive, a weld, or an overmold.
Clause 47: The system of clause 46, where the expandable tip is integral with the distal shaft end.
Clause 48: The system of any one of clauses 41 to 47, further including the occlusion device, where the occlusion device includes an expanding member configured to expand in response to receiving a volume of biogel.
Clause 49: The system of clause 48, where the expanding member includes a device frame, and where the device frame includes at least one of a film layer, a mesh layer, a woven layer, or a non-woven layer.
Clause 50: The system of any one of clauses 41 to 49, where the elongated shaft defines a shaft lumen extending from the proximal shaft end to the distal shaft end, and where the shaft lumen is configured to deliver biogel to the occlusion device to cause expansion of the occlusion device.
Clause 51: The system of clause 50, where the shaft lumen is a first shaft lumen, where the elongated shaft defines a second shaft lumen extending from the proximal shaft end to the distal shaft end, and where the second shaft lumen is configured to retrieve blood from an anatomical chamber in response to the expansion of the occlusion device in the anatomical chamber.
Clause 52: A method including: advancing an elongated shaft including an expandable tip coupled to an attachment collar of an occlusion device through an anatomical structure of a patient to deliver the occlusion device within an anatomical chamber of the patient; expanding the occlusion device to occlude at least a portion of the anatomical chamber; uncoupling the elongated shaft from the occlusion device by releasing the attachment collar of the occlusion device from the expandable tip; and retracting the elongated shaft from the patient.
Clause 53: The method of clause 52, where expanding the occlusion device includes delivering a biogel within an interior of the occlusion device to cause the occlusion device to expand.
Clause 54: The method of clause 52, further including delivering a biogel within an interior of the occlusion device after expanding the occlusion device.
Clause 55: The method of clauses 53 or 54, where delivering the biogel includes transporting the biogel from a biogel source through a shaft lumen of the elongated shaft to the interior of the occlusion device.
Clause 56: The method of any of clauses 52 to 55, further including withdrawing blood from the anatomical chamber.
Clause 57: The method of clause 56, where the shaft lumen of the elongated shaft is a first shaft lumen, and where withdrawing the blood includes transporting the blood from the anatomical chamber through a second shaft lumen of the elongated shaft.
Clause 58: The method of any of clauses 52 to 57, where the anatomical chamber is a left atrial appendage.
Clause 59: The method of any of clauses 52 to 58, further including, before the advancing: introducing the expandable tip secured to the elongated shaft within an interior of the attachment collar of the occlusion device; and coupling the elongated shaft to the occlusion device by advancing a fill tube through the expandable tip to cause the expandable tip to contact the interior of the attachment collar of the occlusion device.
Clause 60: The method of clause 59, where coupling the elongated shaft to the occlusion device includes radially expanding the expandable tip within the interior of the attachment collar, and where uncoupling the elongated shaft from the occlusion device includes radially contracting the expandable tip to release the attachment collar of the occlusion device.
Clause 61: An occlusion device delivery system including: an elongated shaft extending from a proximal shaft end to a distal shaft end; a clamp assembly secured to the distal shaft end and biased to radially expand in absence of an external constraint; and a coupling sleeve extending at least partly along the elongated shaft, where the coupling sleeve is movable relative to the clamp assembly, where the coupling sleeve is configured to removably couple the elongated shaft to the occlusion device by causing the clamp assembly to contract radially inward to clamp a portion of the occlusion device, and where the elongated shaft is configured to deliver the occlusion device to an anatomical chamber.
Clause 62: The system of clause 61, where the elongated shaft includes at least one polymer, where the clamp assembly includes at least one of a metal, an alloy, or a polymer, and where the coupling sleeve includes at least one of a metal, an alloy, or a polymer.
Clause 63: The system of clauses 61 or 62, where the anatomical chamber is a left atrial appendage.
Clause 64: The system of any of clauses 61 to 63, where the clamp assembly includes a plurality of prongs biased to expand radially outward in the absence of the external constraint.
Clause 65: The system of clause 64, where each prong of the plurality of prongs include a beaded tip.
Clause 66: The system of any of clauses 61 to 65, where the clamp assembly is secured to the distal shaft end by at least one of an adhesive, a weld, or an overmold.
Clause 67: The system of any of clauses 61 to 66, where the portion of the occlusion device includes an attachment collar.
Clause 68: The system of any one of clauses 61 to 67, further including the occlusion device, where the occlusion device includes an expanding member configured to expand in response to receiving a volume of biogel.
Clause 69: The system of clause 68, where the expanding member includes a device frame, and where the device frame includes at least one of a film layer, a mesh layer, a woven layer, or a non-woven layer.
Clause 70: The system of any one of clauses 61 to 69, where the elongated shaft defines a shaft lumen extending from the proximal shaft end to the distal shaft end, and where the shaft lumen is configured to deliver biogel to the occlusion device to cause expansion of the occlusion device.
Clause 71: The system of clause 70, where the shaft lumen is a first shaft lumen, where the elongated shaft defines a second shaft lumen extending from the proximal shaft end to the distal shaft end, and where the second shaft lumen is configured to retrieve blood from an anatomical chamber in response to the expansion of the occlusion device in the anatomical chamber.
Clause 72: A method including: advancing an elongated shaft including a coupling sleeve covering a clamp assembly coupled to a portion of an occlusion device through an anatomical structure of a patient to deliver the occlusion device within an anatomical chamber of the patient; expanding the occlusion device to occlude at least a portion of the anatomical chamber; uncoupling the elongated shaft from the occlusion device by releasing the portion of the occlusion device from the clamp assembly; and retracting the elongated shaft from the patient.
Clause 73: The method of clause 72, where expanding the occlusion device includes delivering a biogel within an interior of the occlusion device to cause the occlusion device to expand.
Clause 74: The method of clause 72, further including delivering a biogel within an interior of the occlusion device after expanding the occlusion device.
Clause 75: The method of clauses 73 or 74, where delivering the biogel includes transporting the biogel from a biogel source through a shaft lumen of the elongated shaft to the interior of the occlusion device.
Clause 76: The method of any of clauses 72 to 75, further including withdrawing blood from the anatomical chamber.
Clause 77: The method of clause 76, where the shaft lumen of the elongated shaft is a first shaft lumen, and where withdrawing the blood includes transporting the blood from the anatomical chamber through a second shaft lumen of the elongated shaft.
Clause 78: The method of any of clauses 72 to 77, where the anatomical chamber is a left atrial appendage.
Clause 79: The method of any of clauses 72 to 78, where uncoupling the elongated shaft from the occlusion device includes retracting the coupling sleeve from the clamp assembly to allow the clamp assembly to expand and release the portion of the occlusion device.
Clause 80: The method of any of clauses 72 to 79, further including, before the advancing: positioning the clamp assembly secured to an elongated shaft about the portion of the occlusion device; and coupling the elongated shaft to the occlusion device by advancing the coupling sleeve over the clamp assembly to cause the clamp assembly to clamp the portion of the occlusion device.

Various examples of the disclosure have been described. Any combination of the described systems, operations, or functions is contemplated. These and other examples are within the scope of the following claims.

1. An occlusion device delivery system comprising:
an elongated shaft extending from a proximal shaft end to a distal shaft end; and
a threaded bushing secured to the distal shaft end,
wherein the threaded bushing is configured to removably couple the elongated shaft to a threaded coupling member of an occlusion device, and
wherein the elongated shaft is configured to deliver the occlusion device to an anatomical chamber.

2-15. (canceled)

16. The system of claim 1, wherein the elongated shaft comprises at least one polymer, and wherein the threaded bushing comprises at least one of a metal, an alloy, or a polymer.

17. The system of claim 1, wherein the anatomical chamber is a left atrial appendage.

18. The system of claim 1, wherein the threaded bushing is configured to be threaded within an interior of the threaded coupling member of the occlusion device.

19. The system of claim 1, wherein the threaded bushing is configured to be threaded about an exterior of the threaded coupling member of the occlusion device.

20. The system of claim 1, wherein the threaded bushing is secured to the distal shaft end by at least one of an adhesive, a weld, or an overmold.

21. The system of claim 1, further comprising the occlusion device, wherein the occlusion device comprises an expanding member configured to expand in response to receiving a volume of biogel.

22. The system of claim 21, wherein the expanding member comprises a device frame, and wherein the device frame comprises at least one of a film layer, a mesh layer, a woven layer, or a non-woven layer.

23. The system of claim 1, wherein the elongated shaft defines a shaft lumen extending from the proximal shaft end to the distal shaft end, and wherein the shaft lumen is configured to deliver biogel to the occlusion device to cause expansion of the occlusion device.

24. The system of claim 23, wherein the shaft lumen is a first shaft lumen, wherein the elongated shaft defines a second shaft lumen extending from the proximal shaft end to the distal shaft end, and wherein the second shaft lumen is configured to retrieve blood from an anatomical chamber in response to the expansion of the occlusion device in the anatomical chamber.

25. A method comprising:
advancing an elongated shaft comprising a threaded bushing coupled to a threaded coupling member of an occlusion device through an anatomical structure of a patient to deliver the occlusion device within an anatomical chamber of the patient;
expanding the occlusion device to occlude at least a portion of the anatomical chamber;
uncoupling the elongated shaft from the occlusion device by unscrewing the threaded bushing from a threaded coupling member of the occlusion device; and
retracting the elongated shaft from the patient.

26. The method of claim 25, wherein expanding the occlusion device comprises delivering a biogel within an interior of the occlusion device to cause the occlusion device to expand.

27. The method of claim 25, further comprising delivering a biogel within an interior of the occlusion device after expanding the occlusion device.

28. The method of claim 27, wherein delivering the biogel comprises transporting the biogel from a biogel source through a shaft lumen of the elongated shaft to the interior of the occlusion device.

29. The method of claim 25, further comprising withdrawing blood from the anatomical chamber.

30. The method of claim 29, wherein the shaft lumen of the elongated shaft is a first shaft lumen, and wherein withdrawing the blood comprises transporting the blood from the anatomical chamber through a second shaft lumen of the elongated shaft.

31. The method of claim 25, wherein the anatomical chamber is a left atrial appendage.

32. The method of claim 25, further comprising, before the advancing, coupling the occlusion device to the elongated shaft by screwing the threaded bushing to the threaded coupling member of the occlusion device.

33. The method of claim 32, wherein coupling the elongated shaft to the occlusion device comprises screwing the threaded bushing within an interior of the threaded coupling member of the occlusion device.

34. The method of claim 33, wherein coupling the elongated shaft to the occlusion device comprises screwing the threaded bushing about an exterior of the threaded coupling member of the occlusion device.
